(19) United States
(12) Patent Application Publication
(10) Pub. No.: US 2026/0171195 A1
(43) Pub. Date: Jun. 18, 2026

(54) YIELD PREDICTION SIMULATION SYSTEM AND METHOD IN CHEMICAL PROCESS

(71) Applicant: SK GAS CO., LTD., Seongnam-si, Gyeonggi-do (KR)

(72) Inventors: Ung Gi HONG, Seongnam-si, Gyeonggi-do (KR); Sung Joo YEO, Seongnam-si, Gyeonggi-do (KR); Seung Hwan KONG, Seongnam-si, Gyeonggi-do (KR); Hae Bin SHIN, Seongnam-si, Gyeonggi-do (KR); Sanghyeon PARK, Seongnam-si, Gyeonggi-do (KR); Tae Hyeop KIM, Seongnam-si, Gyeonggi-do (KR)

(73) Assignee: SK GAS CO., LTD., Seongnam-si, Gyeonggi-do (KR)

(21) Appl. No.: 19/102,751

(22) PCT Filed: Aug. 1, 2023
(86) PCT No.: PCT/KR2023/011211

§ 371 Date: Feb. 12, 2026

(30) Foreign Application Priority Data
Aug. 10, 2022 (KR) 10-2022-0100253

(51) Int. Cl. G16C 20/30; G16C 20/10

(52) U.S. Cl. CPC G16C 20/30; G16C 20/10

(57) ABSTRACT

A yield prediction simulation method for predicting yield of a second cycle based on data for predicting yield of a first cycle in a chemical process, includes preprocessing first cycle data, dividing the first cycle into a plurality of segments based on the preprocessed data, and modeling a yield prediction model for each of the plurality of divided segments to predict the yield of the second cycle.

### CROSS-REFERENCE TO PRIOR APPLICATIONS

This Application is a National Stage Patent Application of PCT International Application No. PCT/KR2023/011211 (filed on Aug. 1, 2023) which claims priority to Korean Patent Application No. 10-2022-0100253 (filed on Aug. 10, 2022), which are all hereby incorporated by reference in their entirety.

### BACKGROUND

The present disclosure relates to yield prediction of a chemical process, and more specifically, to a yield prediction simulation system and method capable of predicting the yield of a second cycle period based on process operation data of a first cycle period and simulating the yield prediction by reflecting tag fluctuations.

When performing a series of processes consisting of multiple steps, integrity guarantee and reliability of each process through organic connection are very important. In order to achieve this integrity, the process requires the development of an efficient process management system that can identify the presence or absence of abnormalities and cause diagnosis of each process from input value of major equipment.

In general, most of the past data generated in industrial processes often has a small number of variables and a linear data structure, so sufficient prediction/classification results can be obtained with existing algorithms. However, due to the development of ICT and sensor technology, data with hundreds or thousands of variables began to be generated in the manufacturing process field. In particular, in modern industrial processes such as chemical and manufacturing processes and power plants, data is becoming increasingly larger and more complex due to various efforts to reduce costs and maximize profits while meeting safety, health, and environmental regulations.

Therefore, it is very important to select and manage data that has a great impact on profit creation among such complex and large amounts of data. For example, in commercial chemical processes, process operating conditions affect catalyst activity in the short-term/long-term, and since catalyst activity is directly related to product production, it is very important to predict catalyst activity from a short-term/long-term perspective.

From a short-term perspective, it is necessary to identify changes in catalyst activity according to process operating conditions and optimize operating conditions to improve catalyst activity and increase product production. From a long-term perspective, in commercial chemical processes that use catalysts, catalysts become deactivated as the process is operated, decreasing their activity, and thus require replacement after a certain period of time. Since such catalyst replacement consumes a lot of time and money, it is important to predict future catalyst activity and determine the catalyst life/replacement period.

Therefore, the development of new technologies is required to identify process operating conditions (key factors) that have a great impact on catalyst activity and to reflect the process operation conditions to improve the accuracy of predicting catalytic reaction activity.
(Patent Document 1) Korean Laid-Open Patent No. 10-2018-0029114 (Mar. 20, 2018)
(Patent Document 2) Korean Patent No. 10-2222125 (Mar. 3, 2021)
(Patent Document 3) Korean Laid-Open Patent No. 10-2018-0061769 (Jun. 8, 2018)
(Patent Document 4) Japanese Laid-Open Patent No. 2020-166749 (Oct. 8, 2020)
(Patent Document 5) Korean Laid-Open Patent No. 10-2019-0060547 (Jun. 3, 2019)
(Patent Document 6) Japanese Laid-Open Patent No. 2022-520643 (Mar. 31, 2022)
(Patent Document 7) Korean Patent No. 10-2218287 (Feb. 22, 2021)

### SUMMARY

An object of the present disclosure is to provide a yield prediction simulation system and method capable of predicting yield of a second cycle period based on process operation data of a first cycle period and simulating the yield prediction by reflecting tag fluctuations.

An object of the present disclosure is to provide a yield prediction simulation system and method capable of further improving prediction accuracy over the entire cycle period by dividing the first cycle period into a plurality of segments based on a catalyst lifespan and a yield change according to the catalyst lifespan, and executing key factor analysis, yield prediction, and tag fluctuation analysis for each segment to perform yield prediction simulation for the second cycle period.

Other purposes of the present disclosure are not limited to the purposes mentioned above, and other purposes not mentioned will be clearly understood by those skilled in the art from the description below.

According to one embodiment of the present disclosure, a yield prediction simulation method for predicting yield of a second cycle based on data for predicting yield of a first cycle in a chemical process is disclosed, the yield prediction simulation method including: a step of preprocessing first cycle data; a step of dividing the first cycle into a plurality of segments based on the preprocessed data; and a step of modeling a yield prediction model for each of the plurality of divided segments to predict the yield of the second cycle.

According to one embodiment of the present disclosure, there is disclosed a computer-readable recording medium on which a computer program for executing the yield prediction simulation method according is recorded.

According to the present disclosure, it is possible to predict the yield of the second cycle period based on the process operation data of the first cycle period and simulate the yield prediction by reflecting tag fluctuations.

According to the present disclosure, it is possible to further improve prediction accuracy over the entire cycle period by dividing the first cycle period into a plurality of segments based on the catalyst lifespan and the yield change according to the catalyst lifespan, and executing key factor analysis, yield prediction, and tag fluctuation analysis for each segment to perform yield prediction simulation for the second cycle period.

In addition, according to the present disclosure, key factors according to process operation conditions can be selected and applied to the prediction model to improve the prediction accuracy of catalyst activity changes, and product sales plans and catalyst replacement timing can be determined through accurate catalyst activity prediction, so that the time and cost required for product production and catalyst replacement can be efficiently managed, and product production can be increased by identifying the change in catalyst activity according to process operation conditions and optimizing the operation conditions to improve catalyst activity.

### BRIEF DESCRIPTION OF THE DRAWINGS

FIG. 1 is a block diagram schematically illustrating a yield prediction simulation system according to one embodiment of the present disclosure.

FIG. 2 is a flow chart schematically illustrating the yield prediction simulation method according to one embodiment.

FIG. 3 is a flowchart schematically illustrating a data preprocessing method according to one embodiment.

FIG. 4 is a flow chart schematically illustrating a segment analysis method according to one embodiment.

FIGS. 5 to 10 are diagrams schematically illustrating a segment analysis process according to one embodiment.

FIGS. 11 and 12 are diagrams schematically illustrating a data realization method according to one embodiment.

FIGS. 13 to 15 are diagrams schematically illustrating a method for reflecting a catalyst aging factor according to one embodiment.

FIG. 16 is a schematic diagram illustrating a yield prediction result according to one embodiment.

FIG. 17 is a schematic diagram illustrating a user interface (UI) for tag fluctuation analysis according to one embodiment.

FIG. 18 is a schematic diagram illustrating the results of a yield prediction simulation according to one embodiment.

### DETAILED DESCRIPTION

According to one embodiment of the present disclosure, a yield prediction simulation method for predicting yield of a second cycle based on data for predicting yield of a first cycle in a chemical process is disclosed, the yield prediction simulation method including: a step of preprocessing first cycle data; a step of dividing the first cycle into a plurality of segments based on the preprocessed data; and a step of modeling a yield prediction model for each of the plurality of divided segments to predict the yield of the second cycle.

The above purposes, other purposes, features and advantages of the present disclosure will be readily understood through the following preferred embodiments related to the attached drawings. However, the present disclosure is not limited to the embodiments described herein and may be embodied in other forms. Rather, the embodiments introduced herein are provided so that the disclosed contents can be thorough and complete and so that the spirit of the present disclosure can be sufficiently conveyed to those skilled in the art.

When terms such as first, second, or the like are used in the present specification to describe components, these components should not be limited by these terms. These terms are only used to distinguish one component from another. The embodiments described and illustrated herein also include complementary embodiments thereof.

In the present specification, the singular includes the plural unless specifically stated otherwise in the phrase. The expressions “including,” “consisting of,” and “constituted by” used in the specification do not exclude the presence or addition of one or more other components in addition to the components mentioned.

In the present specification, the term “software” means a technology that moves hardware in a computer, the term “hardware” means a type of device or apparatus (CPU, memory, input device, output device, peripheral device, or the like) that constitutes a computer, the term “step” means a series of processes or operations connected in time series to achieve a given goal, the term “computer program”, “program”, or “algorithm” means a set of commands suitable for processing by a computer, and the term “program recording medium” means a computer-readable recording medium that records a program used to install, execute, or distribute the program.

The terms such as “portion”, “module”, “unit”, “block”, “board”, or the like, used in the present specification to refer to components of the present disclosure may mean a physical, functional, or logical unit that processes at least one function or operation, and which may be implemented by one or more hardware, software, or firmware, or by a combination of hardware, software, and/or firmware.

In the present specification, a “processing unit”, “computer”, “computing device”, “server device”, and “server” may be implemented as a system having an operating system such as Windows, Mac, or Linux, a computer processor, memory, application programs, and a storage device (for example, HDD, SSD). The computer may be, for example, a desktop computer, a laptop, a mobile terminal, or the like, but these are exemplary and not limited thereto. The mobile terminal may be one of a smart phone, a tablet PC, or a mobile wireless communication device such as a PDA.

Hereinafter, the present disclosure will be described in detail with reference to the drawings. In describing specific embodiments below, various specific contents have been written to more specifically describe the present disclosure and help understanding. However, readers who have knowledge of this field enough to understand the present disclosure will recognize that the present disclosure can be used without these various specific contents. In addition, in describing the present disclosure, it is mentioned in advance that parts that are known or commonly used techniques but are not significantly related to the present disclosure will not be described in order to avoid confusion in describing the present disclosure.

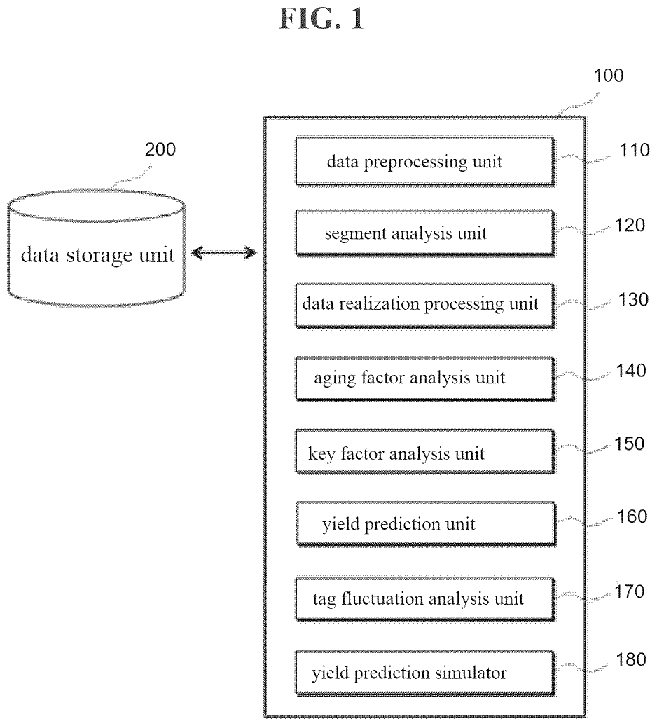

FIG. 1 is a block diagram schematically illustrating a yield prediction simulation system according to one embodiment of the present disclosure. In the following description of the present specification, it is assumed that the system for analyzing a yield prediction simulation system (hereinafter, also simply referred to as a “yield prediction system” or a “simulation system”) according to the present disclosure is applied to an olefin production process. For example, the yield system of the present disclosure may be applied to a Propane DeHydration (PDH) process that produces propylene using propane as a raw material, and through this process, hydrogen may be extracted from propane to produce propylene, which is a type of olefin.

In one embodiment of the present disclosure, the yield prediction simulation system may predict yield of a second cycle period based on process operation data collected during a first cycle period. In this case, the first and second cycles may be periods of the same length of time or periods of different lengths of time, and may be set to, for example, 4 years in the embodiments of the present specification. Preferably, one cycle may be related to the lifespan of a catalyst used in a chemical process, and for example, when the lifespan of the catalyst is 4 years, one cycle may be set to 4 years.

Referring to FIG. 1, a yield prediction simulation system 100 according to one embodiment may include a data preprocessing unit 110, a segment analysis unit 120, a data realization processing unit 130, an aging factor analysis unit 140, a key factor analysis unit 150, a yield prediction unit 160, a tag fluctuation analysis unit 170, and a yield prediction simulator 180, and each of these components (110 to 180) may be implemented as software that is executable and programmed on a computer device, and may also be implemented in part in combination with firmware and hardware, as needed.

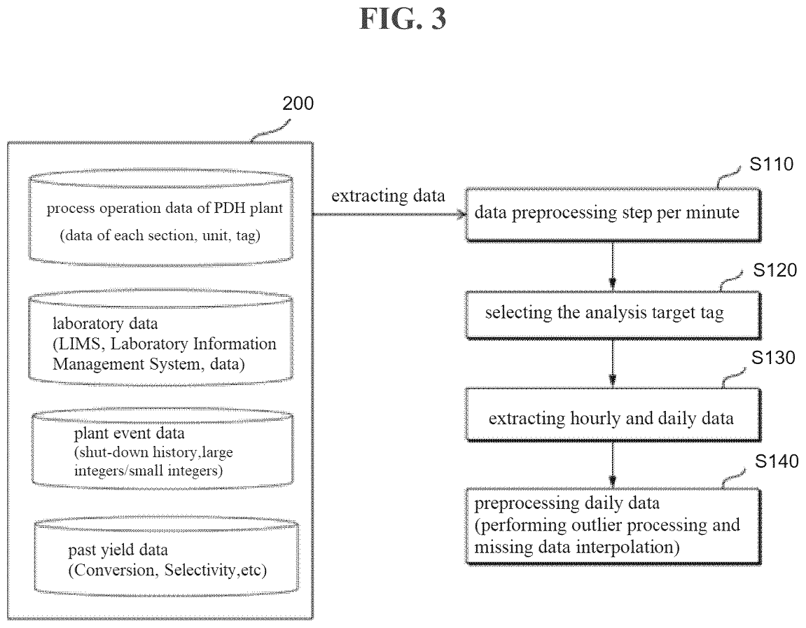

The data preprocessing unit 110 is a functional unit that collects and extracts data from the data storage unit 200 and preprocesses the data. The operation of the data preprocessing unit 110 will be described later with reference to FIG. 3.

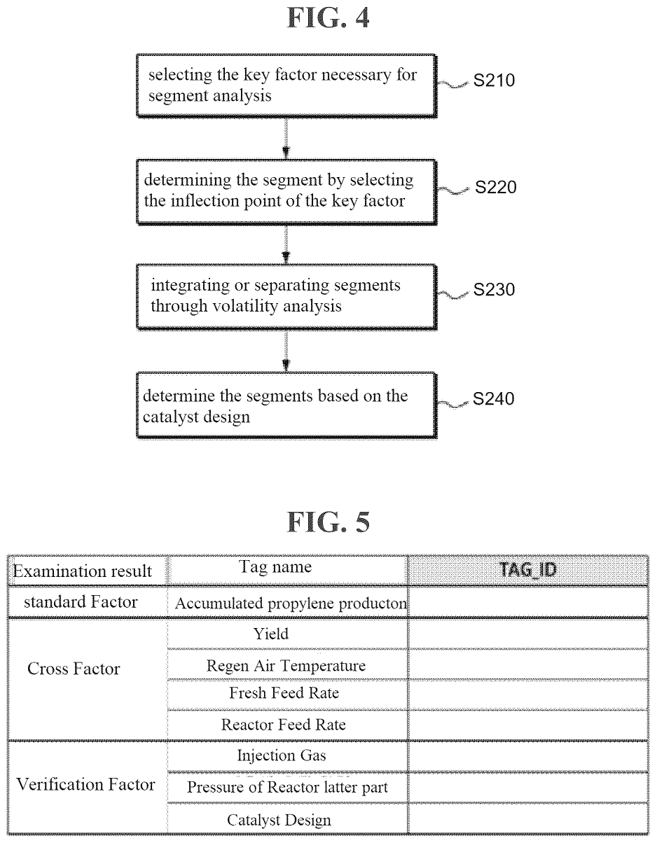

The segment analysis unit 120 may divide one cycle into multiple segments based on the preprocessed data. For example, one cycle is divided into multiple periods based on the amount of change in predetermined factors such as process temperature and yield according to the lifespan (aging) of the catalyst used in the process for one cycle (for example, 4 years). An exemplary operation of the segment analysis unit 120 will be described later with reference to FIGS. 4 to 10.

The data realization processing unit 130 is a functional unit for realizing and generating data used for yield prediction in a form suitable for inputting into the yield prediction model. The yield prediction simulation system according to the present disclosure uses data of a past cycle (the first cycle) to predict a future cycle (the second cycle), and at this time, data of the second cycle may be generated based on the data from the first cycle and input into the yield prediction model.

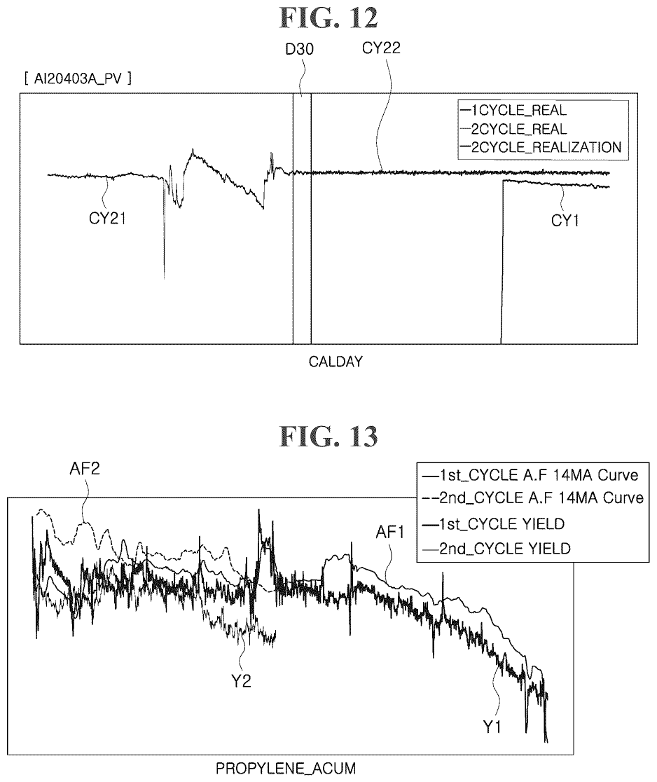

An exemplary operation of the data realization processing unit 130 will be described later with reference to FIGS. 11 and 12.

The aging factor analysis unit 140 is a functional unit for reflecting the aging of a catalyst used in the process in order to more accurately predict the process yield. In general, catalysts have different lifespans depending on their types, and the aging trend may vary within the lifespan. In particular, when the catalyst ages rapidly as it approaches the latter half, it may be difficult to accurately reflect this in the yield prediction model. Therefore, in one embodiment of the present disclosure, the aging factor of the catalyst is additionally considered. For example, the aging factor of the catalyst over time may be calculated and this value may be reflected as a weight in the process data that inputs the yield prediction model, thereby improving the yield prediction performance. An exemplary operation of the aging factor analysis unit 150 will be described later with reference to FIGS. 13 to 15.

The key factor analysis unit 150 extracts the process key factor using the data preprocessed in the data preprocessing unit 110. For example, the key factor analysis unit 150 may be implemented using a known machine learning algorithm, such as a machine learning algorithm that uses a feature selection technique.

The yield prediction unit 160 is a functional unit that predicts the yield of a process using preprocessed data and extracted key factors. In one embodiment, the yield prediction unit 160 may be implemented as a machine learning-based training model, and train the yield prediction model using preprocessed data and key factors, and predict the yield using the trained yield prediction model. For example, when the first cycle data and the first half of the second cycle data are preprocessed, the yield prediction model may be trained using the preprocessed data and key factors, and then the yield prediction result for the remaining period of the second cycle may be output.

The tag fluctuation analysis unit 170 is a functional unit that calculates an amount of change of a tag (input variable input to the yield prediction model). In the present disclosure, the “tag” is the input variable input to the yield prediction model and means various operating conditions such as temperature, pressure, and flow rate in the chemical process, for example. Among the tags, there is a tag (hereinafter, referred to as a “control tag”) that can be controlled and adjusted by a user (for example, a process operator or worker of a plant, or the like), and when the worker changes the tag value of this control tag, the tag values of the remaining tags also change. The tag fluctuation analysis unit 170 may calculate the amount of change of at least some of the remaining tags due to the change in one or more of the control tags, and may be implemented by, for example, a machine learning algorithm trained with process operation data stored in the data storage unit 200.

The yield prediction simulator 180 is a functional unit that simulates a change in yield based on the yield predicted by the yield prediction unit 160 and the tag fluctuation analysis result calculated by the tag fluctuation analysis unit 170. The yield prediction unit 160 statically predicts the yield of the second cycle period based on the data stored so far (data of only the first cycle, or data of the first cycle and data of the first half of the second cycle), whereas the yield prediction simulator 180 may simulate how the future yield changes when the process conditions (temperature, pressure, flow rate, or the like) are changed. To this end, the yield prediction simulator 180 receives the predicted yield calculated by the yield prediction unit 160 and the analysis result calculated by the tag fluctuation analysis unit 170 together and simulates the yield change.

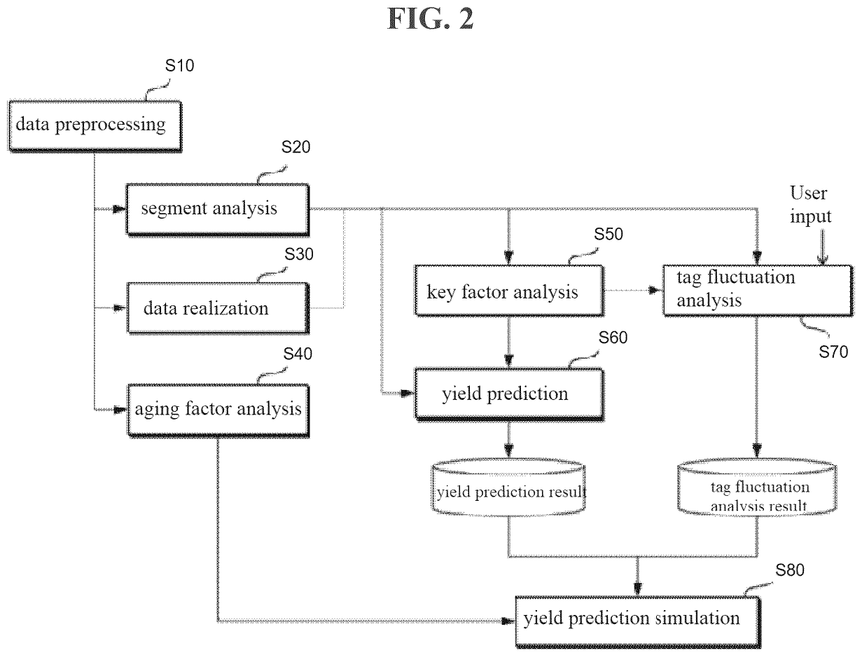

Hereinafter, an exemplary operation of the yield prediction simulation system will be described with reference to FIG. 2. FIG. 2 is a flowchart illustrating the yield prediction simulation method according to one embodiment. Referring to FIG. 2, the yield prediction simulation method according to one embodiment includes a step (S10) of preprocessing data for predicting yield including data of at least the first cycle, a segment analysis step (S20) of dividing one cycle into a plurality of segments based on the preprocessed data, a data realization step (S30) of generating and realizing data of the second cycle based on the first cycle data and the first half data of the second cycle, and an aging factor analysis step (S40) of calculating an aging factor of a catalyst used in a process.

In addition, the yield prediction simulation method includes a step (S50) of analyzing key factors for each segment based on the segment analysis results, and a step (S60) of modeling a yield prediction model for each segment to predict the yield of the second cycle.

In addition, the yield prediction simulation method may further include a tag fluctuation analysis step (S70) of calculating the amount of change of the remaining tags when the user changes the tag value of the control tag, and a yield prediction simulation step (S80) of simulating the amount of change of the yield by reflecting the changed tag values in the predicted yield predicted in the yield prediction step (S60).

Hereinafter, each step of FIG. 2 will be described in more detail. FIG. 3 illustrates an exemplary method of the data preprocessing step (S10) according to one embodiment. Referring to FIG. 3, the data preprocessing step (S10) may include a step (S110) of preprocessing the data for yield prediction in units of minutes, a step (S120) of selecting the analysis target tag, a step (S130) of extracting hourly and daily data for the data of the selected tag among the data preprocessed in units of minutes, and a step (S140) of performing outlier processing and missing data interpolation for the daily data.

In order to preprocess data in the step (S110), data required for yield prediction is collected and extracted from the data storage 200. In this case, the data storage unit 200 may be implemented as a database, for example, but the data format is not particularly limited. In one embodiment, the data for yield prediction extracted from the data storage unit 200 may include (i) process operation data of the olefin production plant, (ii) laboratory data including LIMS data, (iii) plant event data including data regarding time when the plant is not operated normally, and (iv) past yield, conversion rate, and selectivity data regarding olefin production.

The process operation data of an olefin production plant (PDH plant) may be sensor data collected from sensors installed in various facilities of the plant (for example, reactors, pipelines, or the like). Each sensor may be a sensor that measures variables that can observe the process operation status, such as temperature, pressure, flow rate, and composition, and data may be collected from each sensor on a minute-by-minute basis.

The process operation data may be classified by section, unit, and tag and stored in the data storage unit 200. In this case, the unit is a mid-size set of tags within the plant, the section is a large-size set of units, and multiple sections are collected to form the entire PDH plant. Meanwhile, the tag may function as an identifier that identifies each sensor installed in the plant. That is, a unique tag is assigned to each sensor, and for example, when more than 9,000 sensors are installed in the PDH plant, there may be as many tags as the corresponding number. In the following description, unless there is a particular concern of confusion, the data output from the sensor corresponding to each tag is also referred to as a “tag” or “tag data”.

The laboratory data may include Laboratory Information Management System (LIMS) data. In one embodiment, not only actual observation data (tag data) but also laboratory data may be utilized for accurate yield prediction. In addition, the laboratory data may be used to process and interpolate outliers or missing data in tag data. In an alternative embodiment, the laboratory data may be omitted.

The plant event data may include data on times when the plant is not operated normally (shut-down history), large integers/small integers, or the like, and may be used when analyzing and processing the outliers or missing data in the tag data. The past yield value includes past yield data on olefin production. In addition, in this case, the conversion rate (conversion) and selectivity may be included in addition to the past yield. In the following specification, the yield, conversion rate, and selectivity are collectively referred to as a “yield” unless there is concern about confusion, and the yield (that is, yield, conversion rate, selectivity) may be also referred to as a “target” from a machine learning perspective.

In this way, the data stored in the data storage unit 200 may be continuously accumulated in a predetermined set cycle unit and may be data from the past for more than one cycle period from the present time. In this case, the set cycle unit may be a second unit or a minute unit, and for example, data may be collected in 30 second units and then converted to a minute unit and/or an hour unit for analysis and stored. However, such a set cycle unit is exemplary and is not limited to a specific cycle. In addition, one cycle may be set in relation to the catalyst lifespan and may be set to 4 years in one embodiment, but it will be understood that this is exemplary.

The data for yield prediction extracted from the data storage unit 200 is preprocessed as minute unit data in the step (S110). For example, when data in seconds is received from the data storage unit 200, it is converted to minutes, and when outliers or missing data occur, outlier processing and missing data interpolation are performed.

Next, the analysis target tag is selected in the step (S120). For example, key factor analysis and yield prediction, or the like, described below, may be performed using all tag data collected from all sensors installed in the plant, but preferably, some tag data may be selected from the entire tag data, and time/daily data extraction, key factor analysis, and yield prediction (for example, after the step (S130) may be performed using the selected tag data, and in this case, the analysis target tag is selected in the step (S120). For example, in the selection of the tag, the tag recognized as useful for analysis may be selected based on past research and the knowledge and experience of field engineers.

In one embodiment, the analysis target tag selection step (S120) may be performed in advance before the step (S110) of preprocessing minute-unit data, and in this case, the minute-unit data preprocessing step (S110) may be performed only for the tag selected as the analysis target.

In the step (S120), when the analysis target tag is selected, then in the step (S130), time-unit data is extracted and processed again to extract daily data. In this case, for the integration of the data, data integration may be performed by utilizing process data (tag data) and LIMS data. In addition, in an alternative embodiment, the step (S120) of selecting the analysis target tag can be performed after extracting the time-unit data, and in this case, time-unit data may be extracted for all process data and then daily data may be extracted only for the analysis target tag.

After extracting the daily data in the step (S130), the data preprocessing is performed in the step (S140). For example, the data preprocessing includes outlier processing and missing data interpolation. In the case of the outlier processing, only refined values are used as valid input values after selecting and excluding or correcting outliers. In addition, the missing data interpolation is performed for sections selected as outliers and removed or sections without process data due to plant shutdown. The missing data interpolation may be performed by generating new data through, for example, linear regression and distribution-based random number generation.

The yield prediction data preprocessed through the above steps may be organized and converted into a data format to be used in a machine learning training model and then stored in the data storage unit 200 or another arbitrary storage unit.

FIGS. 4 to 10 are diagrams illustrating an exemplary method of the segment analysis step (S20 of FIG. 2) according to one embodiment, FIG. 4 is a flowchart illustrating an exemplary method of the segment analysis step, and FIGS. 5 to 10 are diagrams illustrating a segment analysis process according to one embodiment.

In the segment analysis step (S20), one cycle is divided into multiple segments based on the preprocessed data. In one embodiment, by dividing the segments by considering an inflection point where a yield trend changes rapidly and the degree of yield fluctuation, the segments may be divided into sections showing similar yield increase/decrease trends during the process operation period of one cycle, and by utilizing each segment for various modeling such as subsequent key factor extraction, yield prediction, and yield prediction simulation, the yield prediction accuracy can be increased.

Referring to FIG. 4, in one embodiment, the segment analysis step (S20) may include a step (S210) of selecting the key factor necessary for segment analysis, and a step (S220) of first determining the segment by selecting the inflection point of the key factor. In addition, in one embodiment, after the step (S220), a step (S230) may be further included to secondarily determine the segments by integrating or separating segments through volatility analysis. In addition, in one embodiment, after the step (S230), a step (S240) may be further included to thirdly determine the segments based on the catalyst design.

Briefly explaining each step, first, in the step (S210), the key factor necessary for segment analysis is selected. For example, the key factor may be selected including at least one of the target values such as yield, conversion rate, and selectivity, and the key tags that affect these target values. In this regard, FIG. 5 exemplarily illustrates eight key factors selected in the step (S210).

Next, in the step (S20) of dividing into segments, the inflection point of the key factor is found and analyzed to determine the segment first. . . . As a specific method for finding the segment by analyzing the inflection point, the segment may be determined by (i) calculating the inflection points for one or more factors necessary for segment selection within one cycle, (ii) clustering the calculated inflection points, and (iii) selecting the inflection point that becomes a segment boundary among the clustered inflection points.

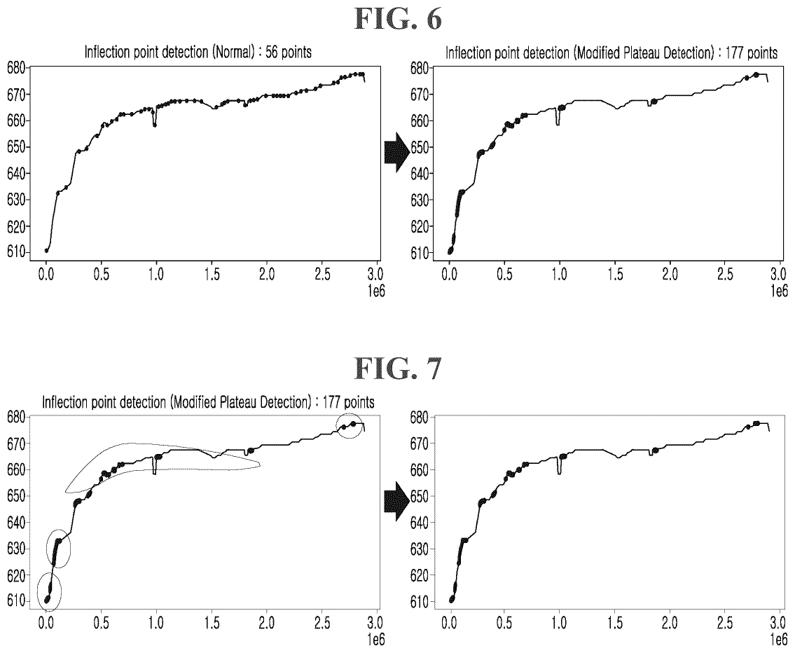

More specifically, in the step (i), the inflection point may be calculated using a known method such as the Plateau Detection method, for example. As an example, FIG. 6 illustrates the result of detecting the inflection point using the modified Plateau Detection method. In FIG. 6, the graph is a graph of air temperature during catalyst regeneration (Regen Air Temperature), and the X-axis represents time (or a variable corresponding to time such as “accumulated production volume”) within one cycle, and the Y-axis represents temperature.

After that, in the step (ii) above, the inflection points are clustered. For example, FIG. 7 illustrates the clustering of inflection points using a Density-Based Spatial Clustering of Applications with Noise (DBSCAN) method that clusters data using the degree of data density. The DBSCAN is one of the known clustering methods, and it goes without saying that the present disclosure is not limited to this method.

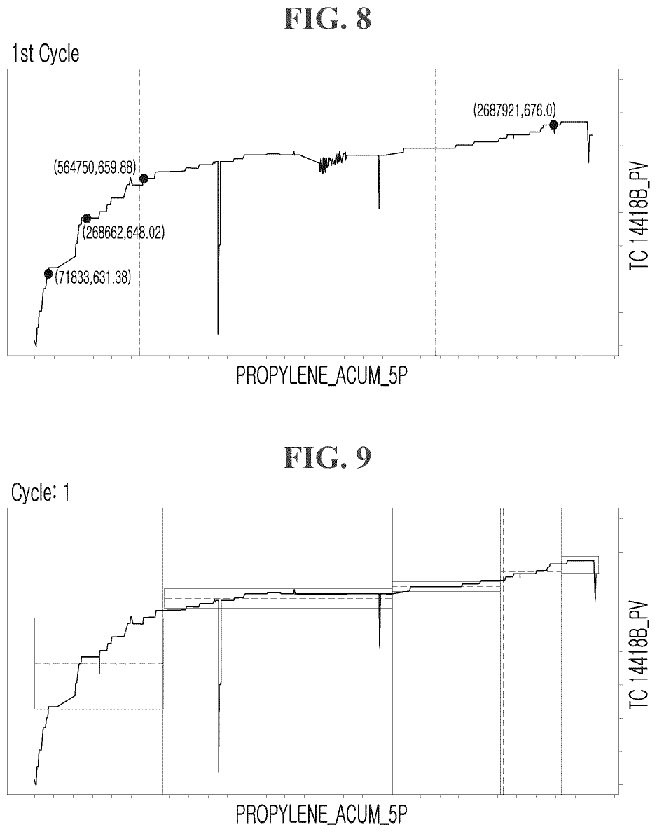

Next, in the step (iii), the inflection point that become the boundaries of the segments is selected among the clustered inflection points to determine the segment first. For example, FIG. 8 illustrates the result of the first segment determination divided into four segments by the step (iii).

In one embodiment, the first determined segment division result as described above may be used to proceed to the next step (for example, the key factor analysis step (S50) of FIG. 2, or the like), and in an alternative embodiment, after the step (S220), the step (S230) of determining the segment for the second time by integrating or separating the segments by volatility analysis may be further included. In the step (S230), for example, a segment-by-segment average and deviation for each of the first determined segments may be calculated, and the segments may be determined secondarily by integrating or separating the first determined segment based on the calculated average and deviation. FIG. 9 illustrates the segment division result determined secondarily by this step (S230). In FIG. 9, the dotted line is the first segment division result by the step (S220), and the blue solid line illustrates the second segment division result by the step (S230) as an example.

In one embodiment, the secondarily determined segment division result as above may be used to proceed to the next step (for example, the key factor analysis step (S50) of FIG. 2, or the like), and alternatively, after the step (S230), the step (S240) of determining the segments thirdly based on the catalyst design may be further included. In the step (S240), for example, the segment division is finally determined by comparing the similarity with the catalyst design, considering the cumulative product production (for example, 600,000 tons, 1.8 million tons, 2.4 million tons, or the like).

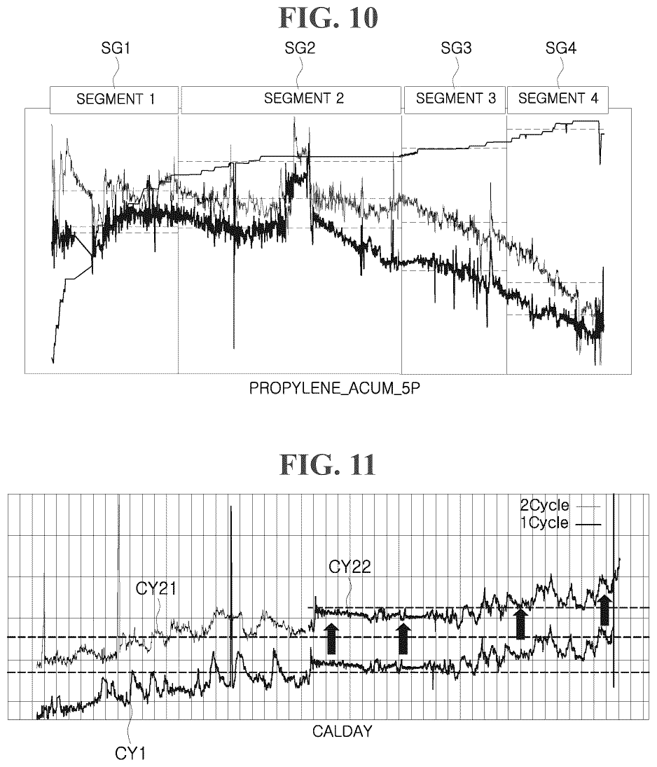

For example, FIG. 10 illustrates the segment division result finally determined by this step (S240). In FIG. 10, when the catalyst lifespan is set to one cycle (for example, 4 years), one cycle is divided into four segments (SG1 to SG4). In FIG. 10, the red graph represents the air temperature (Regen Air temperature) during catalyst regeneration, the gray graph represents the yield, and the light green graph represents the selectivity, respectively.

In FIG. 10, a first segment SG1 is a period in which the catalyst is introduced, the Regen Air temperature gradually increases, and stabilization is gradually realized, and a second segment SG2 is a stabilization period in which the Regen Air temperature is stably maintained and the yield and selectivity are stably achieved. In a third segment SG3, the yield gradually decreases as the catalyst ages. In other words, even when the Regen Air temperature is increased, the yield does not maintain or increase due to the catalyst aging, but the yield gradually decreases. A fourth segment SG4 is the stage where the yield decreases more rapidly, and even when the Regen Air temperature is increased further, the yield and selectivity do not increase any more, but rather decrease rapidly.

In this way, in the present disclosure, the yield increase/decrease trend according to the catalyst lifespan within one cycle period may be considered and segmented into sections showing similar trends, and by performing modeling for each segment in subsequent steps (for example, the key factor analysis step (S50), the yield prediction step (S60), the yield prediction simulation step (S80) of FIG. 2, or the like) for each segment and deriving analysis/prediction results for the entire cycle, the yield prediction accuracy can be improved.

Now, referring to FIGS. 11 and 12, an exemplary method of the data realization step (S30 of FIG. 2) will be described. The data realization step (S30) generates and realizes data for the remaining period of the second cycle based on the first cycle data and the first half of the second cycle data. The yield prediction step (S60) according to the present disclosure uses data from one past cycle (the first cycle) to predict one future cycle (the second cycle). More specifically, data for the second cycle is generated based on data from the first cycle, and then the second cycle data is input into the yield prediction model to predict the yield of the second cycle. In this case, when there is data for a part of the second cycle (hereinafter referred to as the “first half of the second cycle”), the data realization step (S30) generates data for the remaining period of the second cycle by utilizing the data for the first half of the second cycle.

For example, it is assumed that one cycle period is 4 years, the first cycle is from January 2017 to December 2020, and the second cycle is from January 2021 to December 2024. Assuming that the current time point is August 2022, the data storage unit 200 stores the process operation data of the first cycle and the data for the first half of the second cycle (that is, from January 2021 to July 2022).

In this case, the data realization processing unit 130 generates data for the remaining period of the second cycle (that is, from August 2022 to December 2024) based on the process operation data of the first cycle and the data for the first half of the second cycle. In this case, the data realization processing unit 130 may generate the data for the remaining period of the second cycle by considering the characteristics of the data of the first cycle, such as the trend or average, and the characteristics of the data of the first half of the second cycle.

However, the process operation data (for example, each tag data) of the first and second cycles tend to have different trends or values of the two cycles due to differences in catalyst input amount, initial operating conditions, or the like, and therefore, there is a problem that it is difficult to generate data of the second cycle by directly applying the operating condition data of the first cycle. Therefore, in the data realization step S30 of the present disclosure, the data of the first cycle is corrected to suit the trend of the second cycle to generate data of the second cycle.

In one embodiment, the method of realizing each tag data may include at least an average difference reflection method and a random number generation method.

The average difference reflection method may be applied when an average difference exists between the first and second cycles. In one embodiment, when the data within a predetermined period of the first cycle and the second cycle have similar fluctuation trends but different average values, the average point of the first cycle data is moved to generate the data of the second cycle. For example, FIG. 11 illustrates exemplary tag data to which the average difference reflection method can be applied. In FIG. 11, the X-axis corresponds to the time of one cycle, and the Y-axis represents the data value of the corresponding tag. In addition, a black graph is data CY1 of the first cycle of the corresponding tag, and a red graph is data CY21 of the first half of the second cycle. It will be understood that the time point at which data CY2 of the first half of the second cycle ends is the current time point.

When comparing the first cycle data CY1 and the data CY21 of the first half of the second cycle, the trends of the two data are similar, but the average value of the data of the second cycle is larger. Therefore, in this case, the average difference reflection method may be applied to raise the data of the corresponding period of the first cycle by the average difference to generate the data of the remaining period of the second cycle. In this case, in one embodiment, the data of the corresponding period of the first cycle may be used as is with the average raised, or alternatively, the data may be modified by a method such as random number generation for at least some sections to generate the second data.

The random number generation method may be applied when the data of a given period is incomplete or an outlier exists. In one embodiment, when there is incomplete data in the first cycle, a random number is generated to generate the data of the second cycle. For example, FIG. 12 illustrates exemplary tag data to which the random number generation method may be applied.

In FIG. 12, the X-axis corresponds to the time of one cycle, and the Y-axis represents the data value of the corresponding tag. The black graph is the data CY1 of the first cycle of the corresponding tag, and the orange graph is the data CY21 of the first half of the second cycle. The time point when the data CY2 of the first half of the second cycle ends indicates the current time point.

Referring to FIG. 12, the first cycle data CY1 is generated after a certain time point. In other words, the corresponding tag data may mean that the sensor is not installed or the sensor is not operating before the certain time point. However, for the second cycle, there is the first half data CY21, and the remaining section data CY22 generates a random number based on the data of the first cycle to generate the second half data CY22. In this case, for example, at the current time point, the second half data CY22 may be generated by calculating the average and variance of a past predetermined section (for example, 30 days (D30) from the current time point) and maintaining this average and variance while randomly generating data for individual times.

Now, an exemplary method of the aging factor analysis step (S40 of FIG. 2) will be described with reference to FIGS. 13 to 15. The aging factor analysis step (S40) may be performed to reflect the aging of the catalyst used in the process in order to more accurately predict the process yield. In the PDH process for producing propylene, as the cumulative production of propylene increases, the catalyst life decreases, causing the catalyst yield to drop sharply in the latter half of the process. Therefore, it may be desirable to perform the yield prediction by applying catalyst aging factors to accurately reflect yield changes due to changes in process conditions as well as yield decreases due to catalyst life to the yield prediction.

In one embodiment, the aging factor of the catalyst can be indexed as the daily propylene production divided by the amount of heat applied to the catalyst, as in the following Expression, to reflect the yield decrease.

$$AF ( aging factor ) = ( daily propylene production ) / ( amount of heat applied to catalyst )$$

In the above Expression, the “amount of heat applied to the catalyst” may be calculated by multiplying the tag data value indicating the Regen Air temperature by the flow rate, for example.

For example, referring to FIG. 13, a yellow graph represents a yield Y1 of the first cycle over time in one cycle, and a red graph represents an aging factor AF1 of the first cycle. It can be seen that the aging factor AF increases and decreases in a similar trend to the yield Y1. In addition, as explained with reference to FIG. 10, the yield decreases in the last section of the cycle, the fourth segment, no matter how much the temperature is increased. At this time, the yield decreases relatively linearly up to the third segment, but the yield decreases nonlinearly in the fourth segment.

Meanwhile, in FIG. 19, a green graph represents a yield Y2 up to the current time point in the second cycle, a blue graph represents an aging factor AF2 up to the current time point in the second cycle, and each of them progresses in a similar trend to the yield Y2 and aging factor AF1 of the first cycle, so it may be estimated that the yield Y2 also decreases nonlinearly for the fourth segment period. In order to predict this more accurately, the yield is calculated by reflecting the aging factor AF for the second cycle.

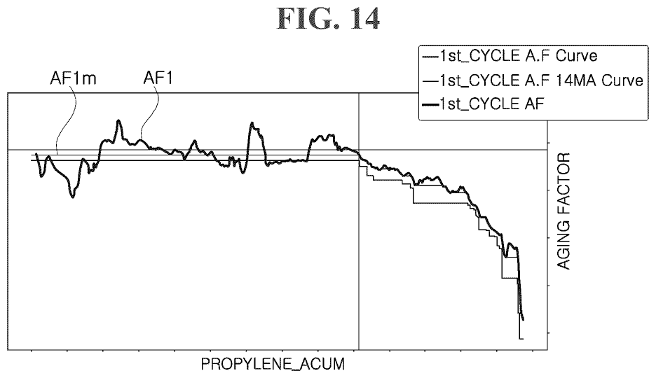

For example, as illustrated in FIG. 14, for the aging factor AF1 of the first cycle, an average value AF1m is first calculated for each segment, and the calculated average value of each segment can be applied as a weight to each segment of the second cycle. In one embodiment, since the yield prediction model predicts the yield relatively accurately even without applying the aging factor AF to the first and second segments, the aging factor AF can be applied to the third and fourth segment sections without applying the aging factor to the first and second segments. In another embodiment, since the yield of the third segment decreases relatively linearly, the yield prediction model can bed predicted with some accuracy, so the aging factor AF can be applied only to the fourth segment.

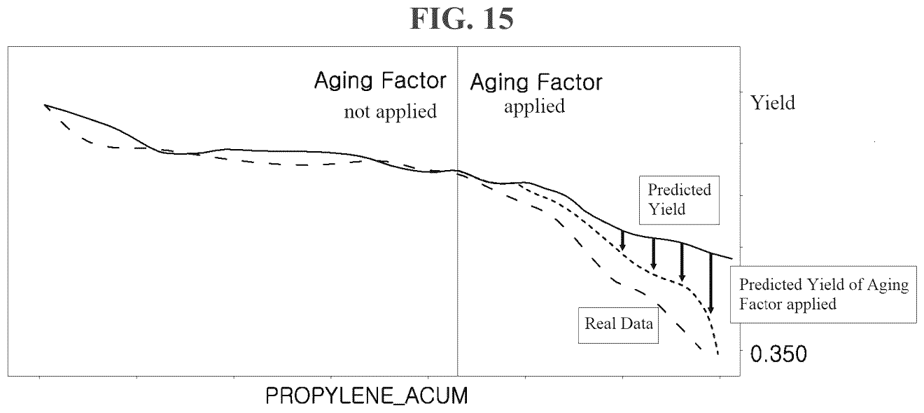

FIG. 15 illustrates an example of the trend of the predicted yield when the aging factor AF is not applied to the first and second segment sections, but only to the third and fourth segments. When the aging factor AF is not applied to the third and fourth segments, the yield is predicted as illustrated in the blue graph in FIG. 15, and the predicted yield (blue solid line) and the actual yield (black dotted line) do not show a large difference in the first and second segments, but a large error is shown in the third and fourth segment sections. However, when the aging factor AF is applied to the third and fourth segments as in the present disclosure, the yield prediction becomes as illustrated in the red dotted line, and a prediction that is relatively close to the actual yield is possible.

Meanwhile, as illustrated in FIG. 2, the results of the aging factor analysis step (S40) may be applied when performing the yield prediction simulation 80. However, in an alternative embodiment, the results of the aging factor analysis may also be applied to the yield prediction step (S60).

Now, referring back to FIG. 2, the key factor analysis step (S50) will be briefly described. The key factor analysis step (S50) extracts process key factors affecting yield by using the data preprocessed in the data preprocessing unit 110. For example, the key factors for the second cycle data generated in the data realization step (S30) may be extracted for each segment divided by the segment analysis step (S20). The key factor extraction method may be implemented as a machine learning algorithm that uses a feature selection technique, for example, a known machine learning algorithm, such as the Boruta algorithm.

The key factors extracted in the step (S50) may be utilized in the yield prediction model in the subsequent yield prediction step (S60). For example, in this step (S50), about 100 key factors are selected from total of about 9,000 tags in the plant, and in the subsequent yield prediction step (S60), yield prediction may be performed based on the tag data values of about 100 key factors selected. In addition, in one embodiment, these key factors may also be utilized in the tag fluctuation analysis step (S70).

Referring to FIG. 2, the yield prediction step (S60) predicts the target of the process (at least one of yield, conversion rate, and selectivity) using preprocessed data and key factors. For example, the yield of the second cycle may be predicted using the second cycle data generated in the data realization step (S30) for each segment divided by the segment analysis step (S20).

For example, the yield prediction model may use a yield prediction model with high predictive power by ensembling a bagging algorithm such as Random Forest and a boosting algorithm such as XGBoost and LightGBM (LGBM), and the model may be trained using the first cycle data as training data.

The bagging series algorithm has the characteristic of increasing training data by repeating random sampling in parallel multiple times and aggregating them. Therefore, even when the training data is insufficient, it provides sufficient learning effects and prevents underfitting and overfitting. The boosting series algorithm also performs random sampling multiple times, but the boosting series algorithm does not perform the random sampling in parallel but sequentially performs the random sampling, and the boosting series algorithm has the characteristic of adjusting the weight of the next training data based on the previous learning results to proceed with learning. In other words, since the boosting series algorithm assigns a high weight to the wrong answer, the boosting series algorithm has the effect of obtaining high accuracy. Among these models, it may be desirable to apply the Random Forest algorithm, which is widely used among the bagging series algorithms that prevent overfitting and underfitting, or the LightGBM model, which is among the boosting series algorithms that train errors to increase accuracy, for modeling. However, this yield prediction model is exemplary, and it goes without saying that a known appropriate machine learning method can be used according to the specific embodiment of the present disclosure.

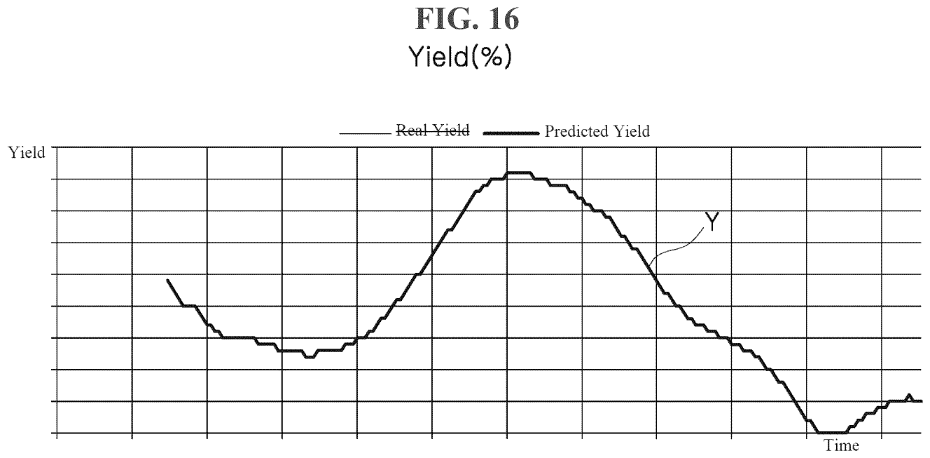

FIG. 16 is an exemplary screen configuration illustrating the results of yield prediction using the ensemble model described above, and in FIG. 16, the predicted yield Y during the second cycle period is illustrated as an orange graph.

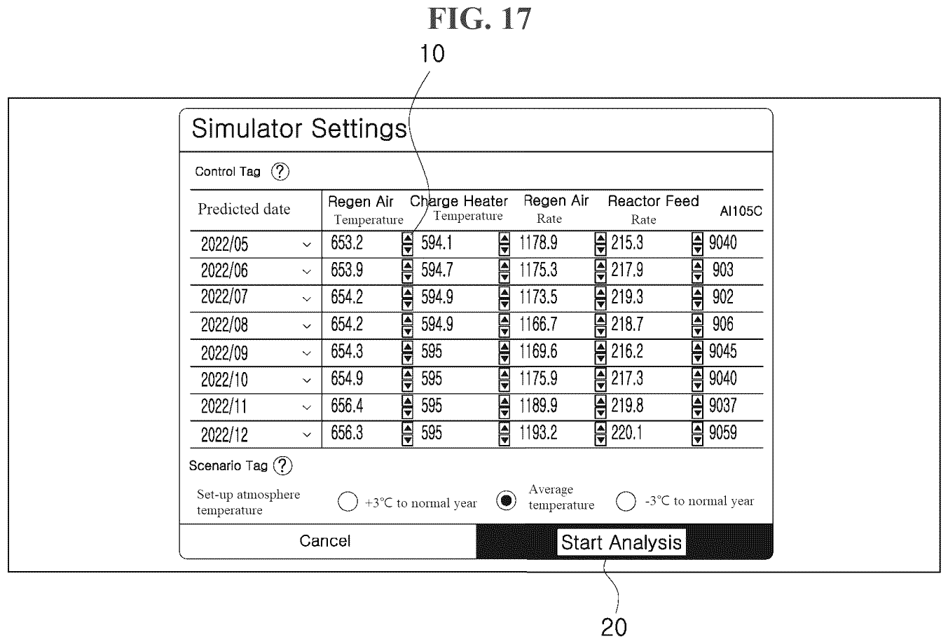

Referring to FIG. 17, the tag fluctuation analysis step (S70 of FIG. 2) will be described. In the tag fluctuation analysis step (S70), the tag variation is analyzed using the tag fluctuation analysis model that calculates the amount of change for at least some of the remaining tags due to the change of one or more control tags among the data for predicting yield.

In one embodiment, at least one control tag that can be manipulated among tags used for yield prediction is selected. Here, the “control tag” means a tag that can be manipulated by a user among process conditions of the chemical process, and may include, for example, at least one tag among air temperature during catalyst regeneration (Regen Air temperature), raw material heating temperature (Charge Heater temperature), air flow rate during catalyst regeneration (Regen Air flow rate), and reactor feed flow rate (Reactor Feed flow rate). Therefore, it will be understood that when, for example, 100 factors (tags) are used for yield prediction, the four tags become control tags and at least some of the remaining 96 tags become tags that change due to changes in the four control tags.

The tag change amount analysis unit 170 may calculate the amount of the change of at least some of the remaining tags due to the change of one or more control tags, and for example, may perform the tag fluctuation analysis (S70) on the tags selected by the second cycle data generated in the data realization step (S30) and the key factor analysis (S50) for each segment divided by the segment analysis step (S20).

In this regard, FIG. 17 illustrates an exemplary user interface (UI) for tag fluctuation analysis. In the embodiment of FIG. 17, it will be understood that four tags are used as the control tags, namely, the air temperature during catalyst regeneration (Regen Air Temperature), the raw material heating temperature (Charge Heater Temperature), the air flow rate during catalyst regeneration (Regen Air Flow Rate), and the reactor feed flow rate (Reactor Feed Flow Rate) are used, and for example, an arrow button 10 may be displayed on the right side of the Regen Air Temperature tag so that the operator can increase or decrease the tag value by adjusting the tag value, and the tag values of the remaining three control tags can also be adjusted with the arrow buttons, respectively. In addition, when the worker adjusts at least one of the four control tags to an arbitrary value and then presses the “Start Analysis” button 20 below (for example, by clicking with the mouse), the tag fluctuation analysis model may calculate and output the amount of change of the remaining tags according to the change in the control tags.

Moreover, the tag fluctuation analysis result performed in the tag fluctuation analysis step (S70) as described above may be used as an input variable in the subsequent yield prediction simulation step (S80). The yield prediction simulation step (S80) is a step for simulating the change in the yield based on the yield predicted by the yield prediction unit 160 and the tag fluctuation analysis result calculated by the tag fluctuation analysis unit 170. In other words, while the yield prediction step (S60) statically predicts the future yield based on the data of the second cycle generated by the data realization (S30), the yield prediction simulation step (S80) dynamically predicts how the future yield changes in a state where the user changes the control tag to an arbitrary value through the tag fluctuation analysis (S70) and the remaining tag values are also changed accordingly. Therefore, in the yield prediction simulation step (S80), the yield prediction simulator 180 simulates the yield change by inputting the predicted yield calculated by the yield prediction unit 160 and the analysis results produced by the tag fluctuation analysis unit 170 together into the yield prediction simulation model.

In addition, as illustrated in FIG. 2, in one embodiment, the yield prediction simulation step (S80) may simulate the change in yield based on the yield predicted by the yield prediction unit 160, the tag fluctuation analysis result calculated by the tag fluctuation analysis unit 170, and the catalyst aging factor calculated by the aging factor analysis step (S40). In this case, the aging factor may be applied as a weight only to the third and fourth segment periods of the second cycle or to the fourth segment period.

The yield prediction simulation model may be implemented with a machine learning algorithm, and for example, a prediction model with high predictive power may be used by ensembling bagging algorithms such as Random Forest and boosting algorithms such as XGBoost and LGBM.

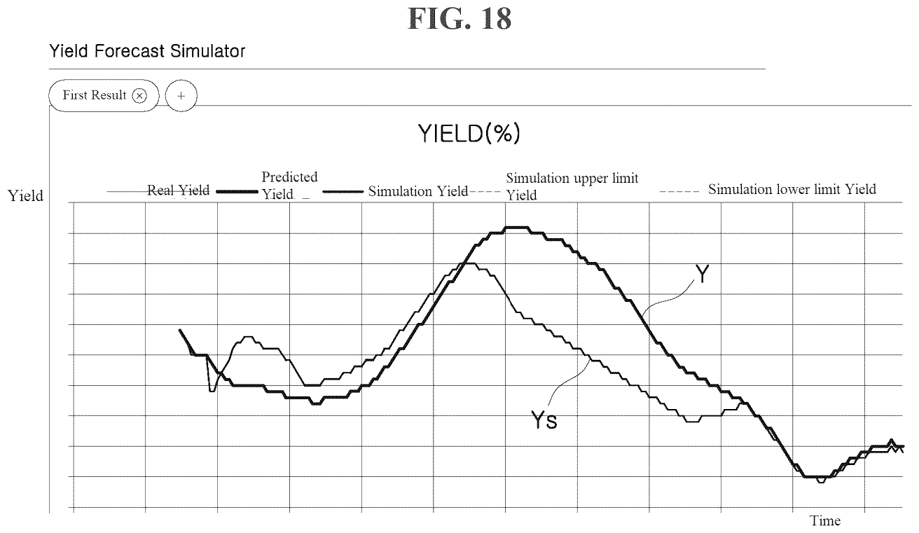

FIG. 18 illustrates an exemplary yield prediction simulation result according to one embodiment. In FIG. 18, an orange graph is the yield Y predicted in the yield prediction step (S60), which is the same as the yield graph of FIG. 16. A pink graph in FIG. 18 is the yield Ys simulated in the yield prediction simulation step (S80). That is, the simulated yield is illustrated in a state where the worker changes at least one control tag value on the screen of FIG. 17 and the remaining tag values are also changed accordingly. Therefore, according to the present disclosure, not only static yield prediction can be performed using the second cycle data, but also the yield analysis due to fluctuations in the main tag can be performed and predicted more accurately and precisely by dynamically simulating how the yield changes due to changes in the control tags when the main control tags are changed.

As described above, anyone with common knowledge in the field to which the present disclosure belongs can understand that various modifications and variations are possible from the description of the present specification. Therefore, the scope of the present disclosure should not be limited to the described embodiments, but should be determined by the claims described below as well as equivalents of the claims.

(DESCRIPTION OF REFERENCE NUMERALS)

| | |
|---|---|
| 100: yield prediction simulation | 110: data preprocessing unit |
| system | |
| 120: segment analysis unit | 130: data realization processing unit |
| 140: aging factor analysis unit | 150: key factor analysis unit |
| 160: yield prediction unit | 170: tag fluctuation analysis unit |
| 180: yield prediction simulator | 200: data storage unit |

1. A yield prediction simulation method for predicting yield of a second cycle based on data for predicting yield of a first cycle in a chemical process, the yield prediction simulation method comprising:
a step (S10) of preprocessing first cycle data;
a step (S20) of dividing the first cycle into a plurality of segments based on the preprocessed data; and
a step (S60) of modeling a yield prediction model for each of the plurality of divided segments to predict the yield of the second cycle.

2. The yield prediction simulation method of claim 1, wherein the first cycle and the second cycle are determined according to a lifespan of a catalyst used in the chemical process.

3. The yield prediction simulation method of claim 1, wherein the step (S20) of dividing into the segments includes a step (S220) of calculating an inflection point for one or more factors necessary for segment selection within the first cycle, clustering the calculated inflection points, and selecting an inflection point that becomes a boundary of the segment among the clustered inflection points to first determine the segment.

4. The yield prediction simulation method of claim 3, wherein the step (S20) of dividing into segments further includes a step (S230) of calculating a segment-by-segment average and deviation for each of the first determined segments, and integrating or separating the first determined segments based on the calculated averages and deviation to determine the segment secondarily.

5. The yield prediction simulation method of claim 1, further comprising a key factor analysis step (S50) of selecting a key factor by modeling a key factor analysis model for each of the plurality of divided segments,
wherein in the yield prediction step (S60), the yield of the second cycle is predicted by using the yield prediction model modeled for each of the plurality of segments based on the preprocessed first cycle data and the key factor derived by the segment-specific key factor analysis.

6. The yield prediction simulation method of claim 5, further comprising a tag fluctuation analysis step (S70) of modeling a tag fluctuation analysis model that calculates an amount of change for at least some of the remaining tags due to a change in one or more control tags among the data for predicting yield, for each of the plurality of divided segments.

7. The yield prediction simulation method of claim 6, wherein the plurality of control tags are tags regarding a process condition adjustable by a user among process conditions of the chemical process.

8. The yield prediction simulation method of claim 6, further comprising a yield prediction simulation step (S80) of simulating the yield of the second cycle based on a predicted yield calculated from the yield prediction model and a tag fluctuation analysis result calculated from the tag fluctuation analysis model,
wherein the tag fluctuation analysis result includes a control tag value arbitrarily changed by a user and tag values of tags output by inputting the control tag value into the tag fluctuation analysis model.

9. A computer-readable recording medium on which a computer program for executing the yield prediction simulation method according to claim 1 is recorded.
